(19) United States
(12) Patent Application Publication
(10) Pub. No.: US 2005/0107874 A1
(43) Pub. Date: May 19, 2005

(54) Sharp angle intraocular lens optic

(76) Inventor: Ehud Assia, Tel Aviv (IL)

Correspondence Address: David Klein;Dekel Patent, Beit HaRofim, 18 Menuha VeNahala Street, Room 27, Rehovot, IL

(21) Appl. No.: 10/507,841

(22) PCT Filed: Mar. 18, 2003
(86) PCT No.: PCT/IL03/00233

Related U.S. Application Data
(60) Provisional application No. 60/364,583, filed on Mar. 18, 2002.

(51) Int. Cl. A61F 2/16

(52) U.S. Cl. 623/6.27

(57) ABSTRACT

An IOL optic is provided having an optic edge with a sharp angle (preferably lower than 90°). The IOL optic has a low rate of capsular opacification, lower than a similar optic with a perpendicular edge or with an edge angle greater than 90°.

### FIELD OF THE INVENTION

The present invention relates generally to intraocular assemblies, and particularly to intraocular assemblies for reducing posterior capsule opacification.

### BACKGROUND OF THE INVENTION

Posterior capsule opacification (PCO) is the most common complication of cataract surgery. Migration and proliferation of lens epithelial cells and transformation to pseudo-fibroblasts are associated with opacification of the posterior capsule and consequently with a decrease in visual acuity and quality. This may require a surgical or laser treatment to create a central opening of the capsule (capsulotomy) to restore vision. The opening of the posterior is associated with potential complications such as retinal breaks and detachment.

Numerous factors related to intraocular lens (IOL) design and configuration have been identified and discussed in the prior art, such as posterior convexity, angulation of the haptic, one-piece design, and lens material. For example, proposals have been made to prevent lens epithelial cell migration using intraocular lenses with sharp rectangular edges or by creating a discontinuous sharp bend in the capsule. The prior art has demonstrated that a square-edge optic (see FIGS. 1B and 2B) is associated with a significant reduction in PCO formation, as compared to a rounded smooth edge (see FIGS. 1A and 2A) which does not provide the barrier effect of the square-edged design. The prior art also contends that the formation of a capsular bend is a significant factor in reducing cell migration towards the center of the posterior capsule. However, the formation of a capsular bend is time-consuming and requires fusion of the anterior and posterior capsules.

### SUMMARY OF THE INVENTION

In the present invention, an IOL optic is provided having an optic edge with a sharp angle (preferably lower than 90°). The IOL optic has a low rate of capsular opacification, lower than a similar optic with a perpendicular edge or with an edge angle greater than 90°. This may be due to the angle created between the capsule and the lens optic, which creates an anatomical or functional barrier for the migrating cells. The bend-effect of the capsule may thus occur early and does not necessarily require fusion of the anterior and posterior capsules.

### BRIEF DESCRIPTION OF THE DRAWINGS

The present invention will be understood and appreciated more fully from the following detailed description, taken in conjunction with the drawings in which:

FIGS. 1A and 2A are simplified side-view and pictorial illustrations, respectively, of rounded smooth edge IOLs of the prior art;

FIGS. 1B and 2B are simplified side-view and pictorial illustrations, respectively, of square-edge IOLs of the prior art;

FIGS. 1C and 2C are simplified side-view and pictorial illustrations, respectively, of a sharp angle IOL, constructed and operative in accordance with an embodiment of the present invention;

FIG. 2D is a simplified pictorial illustration of a sharp angle IOL, constructed and operative in accordance with another embodiment of the present invention;

FIG. 2E is a simplified pictorial illustration of a sharp angle IOL, constructed and operative in accordance with another embodiment of the present invention;

FIG. 3A is a simplified pictorial illustration of a sharp angle IOL, constructed and operative in accordance with another embodiment of the present invention; and

FIG. 3B is a simplified pictorial illustration of a sharp angle IOL, constructed and operative in accordance with another embodiment of the present invention.

### DETAILED DESCRIPTION OF A PREFERRED EMBODIMENT

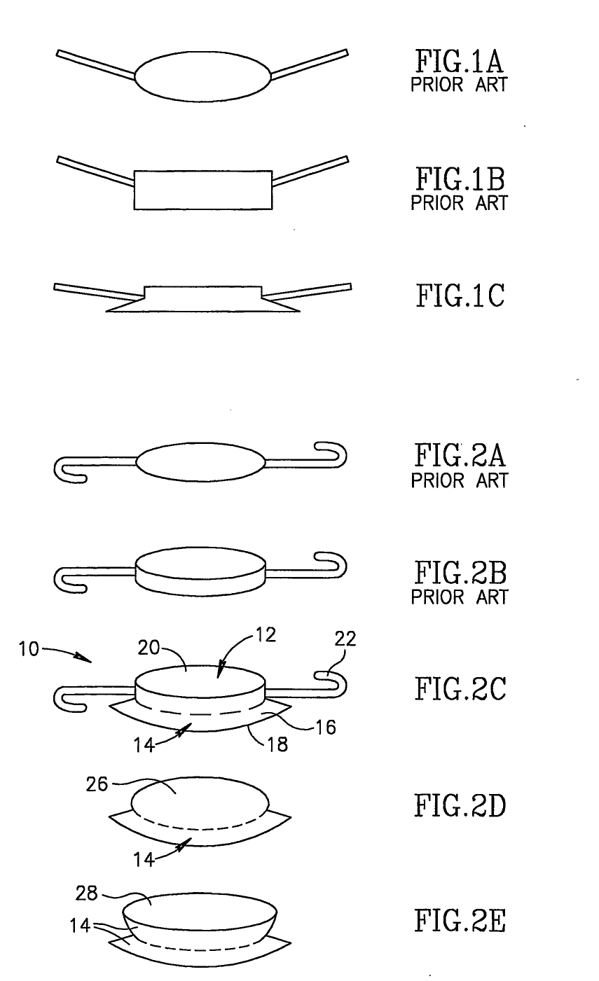

Reference is now made to FIGS. 1C and 2C, which illustrate an IOL 10, constructed and operative in accordance with an embodiment of the present invention.

IOL 10 may comprise a central optic 12 with a sharp-angle extension 14 formed at least partially about a periphery of central optic 12. The sharp-angle extension 14 may comprise two surfaces 16 and 18 that define an acute angle (i.e., not greater than 90°) therebetween

The edge of sharp-angle extension 14 may be sharp or rounded. In FIG. 2C, central optic 12 has a generally flat surface 20 opposite sharp-angle extension 14, and includes haptics 22. In the embodiment of FIG. 2D, a central optic 24 has a generally round surface 26 opposite sharp-angle extension 14. FIG. 2E illustrates a double sharp-edge optic 28 comprising a pair of opposing, generally symmetrical sharp-angle extensions 14. (FIGS. 2D and 2E are shown without the haptics.) The sharp-angle extension 14 may be concave or convex.

The IOL may be either solid (PMMA) or foldable (silicone, acrylic, hydrogel or any other material), for example.

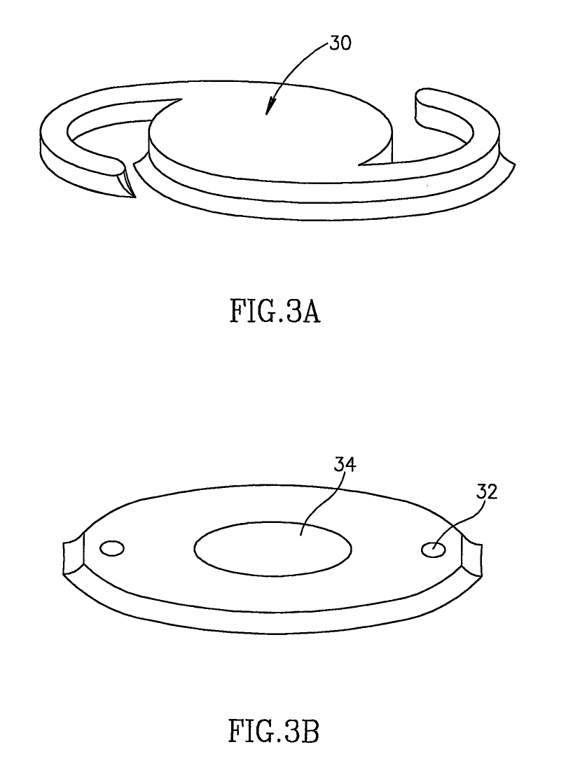

As seen in FIG. 3A, the IOL may comprise a plate-haptic monoblock, one-piece IOL 30 (meaning that the haptics form one piece with the body of the optic). As seen in FIG. 3B, the IOL may be formed with one or more apertures 32 and lenses 34. In general, the IOL of the invention may comprise any shape (e.g., disc shape), may have open or closed loops.

The acute angle may be limited to the optic only, or may also include the lens haptic (as in FIG. 3A). There may or may not be angulation between the optic and the haptic. The optic and haptic may be of any dimension, size and configuration.

The acute angle may be of any magnitude, either minimal (for example 85°) or very acute (for example 30°), yet lower than 90°.

The sharp edge may be of the same material of the optic or an element added to the lens optic.

It is possible that the acute angle may reduce the glare phenomenon, often seen in square edged IOLs.

It will be appreciated by persons skilled in the art that the present invention is not limited by what has been particularly shown and described hereinabove. Rather the scope of the present invention includes both combinations and subcombinations of the features described hereinabove as well as modifications and variations thereof which would occur to a person of skill in the art upon reading the foregoing description and which are not in the prior art.

1. An intraocular lens optic (IOL) comprising a central optic with a sharp-angle extension formed at least partially about a periphery of said central optic, said sharp-angle extension comprising two surfaces that define an acute angle therebetween.

2. The IOL according to claim 1, wherein an edge of said sharp-angle extension is sharp.

3. The IOL according to claim 1, wherein an edge of said sharp-angle extension is rounded.

4. The IOL according to claim 1, wherein said central optic has a generally flat surface opposite said sharp-angle extension.

5. The IOL according to claim 1, wherein said central optic has a generally round surface opposite said sharp-angle extension.

6. The IOL according to claim 1, further comprising a pair of opposing, generally symmetrical sharp-angle extensions.

7. The IOL according to claim 1, wherein said sharp-angle extension is concave.

8. The IOL according to claim 1, wherein said sharp-angle extension is convex.

9. The IOL according to claim 1, further comprising haptics extending from said central optic.

10. The IOL according to claim 9, wherein said haptics comprise a sharp-angle extension.
